(19) United States
(12) Patent Application Publication
(10) Pub. No.: US 2014/0114212 A1
(43) Pub. Date: Apr. 24, 2014

(54) APPARATUS AND METHOD FOR THE ASSESSMENT OF NEURODEVELOPMENTAL DISORDERS

(71) Applicant: University Court of the University of Aberdeen, Aberdeen (GB)

(72) Inventors: Mark Arwyn Mon-Williams, Aberdeen (GB); Justin Hereward Gwilym Williams, Stonehaven (GB); Andrew David Wilson, Leamington Spa (GB); Mandy Suzanne Plumb, Aberdeen (GB)

(73) Assignee: University Court of the University of Aberdeen, Aberdeen (GB)

(21) Appl. No.: 14/056,202

(22) Filed: Oct. 17, 2013

Related U.S. Application Data
(63) Continuation of application No. 12/302,174, filed on Mar. 10, 2009, now abandoned.

(30) Foreign Application Priority Data
May 23, 2006 (GB) 0610259.4
May 23, 2007 (GB) PCT/GB2007/001931

(51) Int. Cl. A61B 5/16; A61B 5/11

(52) U.S. Cl. CPC A61B 5/16; A61B 5/1124
USPC 600/595; 600/300

(57) ABSTRACT

The present invention concerns apparatus for the detection and/or assessment of neurodevelopmental disorders of a user, the apparatus comprising: control means for providing a goal-orientated task to a display, said task being formulated to elicit a movement from a user in response to said task; input means via which a user can input a response to said task; wherein said control means records a user's response as multi-dimensional input data, which can be used to profile the user for the presence or absence of a neurodevelopmental disorder, the task being specifically formulated such that indicative characteristics of a user's movement over time can be identified by said multi-dimensional input data.

The present invention relates to an apparatus and a method for the detection and/or assessment of neurodevelopmental disorders, particularly those presenting in children. This can be used in a wide range of settings including schools.

About 10% of children are affected by mental health problems. Neurodevelopmental impairment is frequently a major contributory factor, resulting in a broad range of disabilities that include autistic spectrum disorder (ASD), attention deficit hyperactivity disorder (ADHD) and developmental coordination disorder (DCD). Unfortunately, the assessment of these children is a time-intensive process. Specialist skills are required to administer and analyse the results of a wide range of tests and the demand for assessment currently outstrips the availability of skills to manage it.

Current tests for assessing neurodevelopmental disorders in children can be categorized according to their objectives. Some assessments are diagnostic. Psychiatric diagnoses are made according to the presence of behavioural symptoms and therefore utilize a range of measures including self-report questionnaires, clinical interviews and behavioural observation. These assessments require high levels of training and are highly time-consuming. Other assessments are designed to assess abilities. Abilities, and disabilities that arise from neurodevelopmental disorders (that might include reading, mathematical or motor skills including handwriting ability for example), can be gauged through psychometric assessments where performance on set tasks can be measured. Such tests may also inform the assessment of behavioural (psychiatric) disorders in children such as ADHD and autism, because behavioural symptoms in these conditions arise secondary to disabilities with skills such as attentional regulation and control over motor skills. A test that can assess attention or goal-directed action will therefore considerably enhance understanding of the disability associated with psychiatric disorder, and will be sensitive in its detection. Psychometric assessments are usually administered as ‘pen and paper’ tests (i.e. they require subjective recording by the examiner), such as the Psychcorp Movement ABC (Assessment Battery for Children). These tests use physical challenges to assess, for example, manual dexterity, ball skills and dynamic balance, with a qualified observer making notes on paper. Such tests require a highly trained professional to collect and analyse the data, and are expensive and time-consuming. They are also highly subjective and therefore open to error.

Other known tests include the CANTAB test battery, a computer automated battery of neuropsychological tests. However, this test focuses on simple measures of behaviour at only isolated time points (choices measured by position touched on a touch-sensitive screen and time taken to depress a switch). Hence these tests rely on simple cognitive tasks and are not sensitive enough to detect impairments in attention and goal directed action that become manifest in the way that an action is performed rather than simply the time taken to perform it or the choices made. Furthermore, there are no data or claims made for the CANTAB that it can contribute to the assessment of DCD.

There is a need to provide more efficient assessment batteries for the detection and appraisal of neurological problems particularly in neurodevelopmental disorders in children that can produce standardized and reliable data for the purpose of measuring ability, that can be employed readily within health and educational settings, that can screen for disorder without the need for highly trained professionals, that can accurately and reliably identify neurodevelopmental disability and that can support behavioural assessments.

According to one aspect of the present invention, there is provided an apparatus for the detection and/or assessment of neurodevelopmental disorders of a user, the apparatus comprising:
control means for providing a goal-orientated task to a display, said task being formulated to elicit a movement by a user in response to said task;
input means via which a user can input a response to said task;
wherein said control means records a user's response as multi-dimensional input data, which can be. used to profile the user for the presence or absence of a neurodevelopmental disorder, the task being specifically formulated such that indicative characteristics of a user's movement over time can be identified by said multi-dimensional input data.

The data taken from the input means can be multi-dimensional data with the tasks being formulated so as to optimise the value or nature of such data. It this regard, multi-dimensional means “in two or more dimensions”. Said dimensions are not limited to spatial dimensions, but may comprise, for example, temporal and force dimensions.

According to another aspect of the present invention, there is provided an apparatus for the detection and/or assessment of neurodevelopmental disorders of a user, the apparatus comprising:
control means for providing a goal-orientated task to a display, said task being formulated to elicit a movement from a user in response to said task;
   input means via which a user can input a response to said task;
   wherein said control means records a user's response as two- and/or three-dimensional input data, which can be used to profile the user for the presence or absence of a neurodevelopmental disorder, the task being specifically formulated such that indicative characteristics of a user's movement over time can be identified by said two- and/or three-dimensional input data.

Preferably, the task is formulated to elicit a three-dimensional movement by a user. Preferably, the control means records a user's response as two-dimensional input data. However, further dimensions are possible, such as time, and/or application pressure of a user's input means, for example that of a stylus on a tablet screen.

Examples of indicative characteristics that can be identified by these data include speed, path length (directedness), smoothness (jerk), and accuracy plus the variability of these variables. Movement perturbations in relation to dynamic visual stimuli (such as video-clips) can also be identified.

The goal-oriented tasks are designed to place demands upon, and thereby test, a wide range of the user's social, cognitive and motor abilities. In this respect, children with neurodevelopmental disorders show deficits in specific areas of behavioral control and patterns of goal-directed actions. This means that different task constraints elicit different impairments of movement pattern depending on the underlying problems. experienced by the children. Some observations on patterns in human movement axe:

(i) If there is an increase in uncertainty in the goal-oriented task (for example, if the movement of a target is unstable or if the precision requirement to fulfill the'task is increased), movements are made correspondingly more slowly. Clinical populations, such as children with DCD, exhibit slower, more variable and less accurate patterns of movement;
(ii) Switching from one task to another requires both the ability to detect the new task and to leave the old task. There is growing evidence that one of the core deficits in ASD is problems with the latter. The goal-oriented task displayed by the apparatus of the present invention could distinguish between the options;
(iii) Children with DCD show slower and less accurate baseline movements that show decreased responsiveness to target shifts;
(iv) Children with ASD show a lesser propensity to use secondary sources of information (especially if cues are social in nature) in order to improve their performance;
(v) Children with ADHD show increased errors and more exaggerated responses to partial cues and/or distracter targets.

The indicative characteristics are preferably based on a library of known calibrated results from tasks provided to children having known disorders.

The control means preferably comprises a computer, more preferably a portable computer such as a tablet laptop computer.

Preferably, the display comprises a computer monitor or a television screen. Preferably, the display comprises a LCD or plasma screen.

The input means can be any input means which monitor a user's movement over time and generate the dimensional data of interest. Suitable input means are hand-held manipulanda such as a mouse, a stylus, a joystick or other hand-held controller. In certain embodiments, the display may be an interactive tablet-screen display, in which embodiments the display means doubles as the input means. The apparatus may comprise more than one input means.

Most preferably, the input means is a hand-held manipulandum (e.g. a stylus) or an interactive tablet-screen display.

The user's 3-D response to tasks is preferably recorded as a function of time to build up a user profile.

In a preferred embodiment, the control means and display means comprise a laptop computer with an integral tablet-screen monitor. The computer runs software which is designed to present the user with a visual and/or aural goal-oriented task. The user then uses the response input means to input his/her 3-D response to the task or stimulus. This response is recorded by the apparatus and analysed using suitable software, to build up, a profile of the user and to aid in the identification and diagnosis of a neurodevelopmental disorder.

The position of the input means (for example a stylus) in spatial coordinates is recorded as the user moves the manipulandum over the display. The data are preferably recorded as a function of time. The data as such allow the measurement of standard movement parameters such as position, velocity and acceleration, as well as the timing characteristics of the movements (e.g. reaction time to movement initiation upon stimulus presentation, movement duration) and the relation between these characteristics (e.g. when changes in position or velocity occur during a movement, smoothness of movement, accuracy).

In preferred embodiments, the apparatus has a secondary function as a practice tool to enable children to practise their responses on skills they find difficult, and thus the apparatus also has a potential therapeutic application.

The apparatus of the present invention can provide a quantitative measurement of behaviour from reaction time through velocity, acceleration and jerk characteristics to the accuracy and variability recorded at the end of the goal-directed movement.

The apparatus of the present invention is self-contained, the data compiled are objective and standardized (such that if two people test the same child they should obtain the same results), and the apparatus is fast to use and can be implemented by non-specialist personnel.

In certain embodiments of the present invention, said control means comprises a database comprising normative data collected from a plurality of users' previous responses to specified tasks using an apparatus according to the present invention, said normative data being for comparison with data generated by a user of said apparatus for the purpose of diagnosing a neurodevelopmental disorder.

According to a further aspect of the present invention, there is provided an apparatus for the detection and/or assessment of neurodevelopmental disorders in children, the apparatus comprising a display for displaying objective orientated information; an input for allowing input of information by a user for reaching an objective; and control means for assessing, from characteristics of said input information in collaboration with said objective orientated information, a type of neurodevelopmental disorder.

According to another aspect of the present invention, there is provided a method of assessing the presence of a neurological disability in a child, which comprises the use of an apparatus as defined above.

In preferred embodiments, the control means generates further tasks on the basis of the user's response to an initial task or tasks. In this way, the apparatus can ‘learn’ to direct tasks more specifically so as to more accurately identify disorders of the user. For example, the user may progress through a series of tasks that change in difficulty as a function of the user's performance (an “adaptive psychophysical staircase technique”). This can be used to establish, for example, a threshold value at which some change or stimulus can be detected. In essence, a task can be made more difficult if the user's performance at a preceding task is satisfactory, or can be made easier if the user's previous performance is non-satisfactory. The degree of increased/reduced difficulty may be varied, as may be the number of tasks required to trigger a change. Tasks may also be tailored as a function of the user's performance. For instance, if the user's behaviour is suggestive of, for example, ASD rather than DOD, the apparatus may automatically present the user with an ASD-relevant task, rather than merely the next task in a static list. Both of these procedures are designed to efficiently establish a profile of the user in question.

### EXAMPLES

In one example, there is displayed on a computer screen a start location and a target location (the goal-oriented task). The child's task is to move the manipulandum from the start location to the target location across the screen. In doing so, the child must move their arm to engage the stylus on the start location, and must then trace the stylus over to the target location. This involves 3-D movement by the child constrained to 2-D, which is recorded via the manipulandum as a change in, spatial location of the manipulandum's endpoint over time. The stimulus could be varied by changing, for example, the number of potential target locations on the screen or the area of the target (the smaller the target, the more difficult the task). Additionally, the target may not be on screen initially, but may suddenly appear and/or may move about the screen during the task duration. Measurements such as reaction speed, accuracy of pin-pointing the start location, movement-path length, movement speed variability, speed, smoothness and accuracy in reaching the target location are recorded with high precision. In this way, a normative database is created against which future data can be compared. Outlying values in any of the measured parameters such as variable movement speed, or poor accuracy relative to the established normal range of variability could be indicative of a neurodevelopmental disorder.

Another example of a goal-oriented task displayed by the apparatus of the present invention is a maze through which a child must trace an escape route. The advantage of recording the user's response as a function of time for a task such as this is that account is taken of the user's “thinking time”. It has been found that users plan the escape route in advance before inputting their response. Thus, the time taken by the user to commence and to complete the task is vital information which prior art “pen and paper” tests (which record only the accuracy of the response) cannot record.

A further example of a goal-oriented task displayed by the apparatus of the present invention is a video clip of an actor looking straight ahead. A coloured dot jumps randomly between corners of the display screen. The user's task is to follow the dot each time it jumps. Three different situations are designed to elicit response data from the user: in the first situation, the actor maintains his straight-ahead gaze; in the second situation, the actor's gaze shifts to the corner of the screen where the dot is about to appear; in the third situation the actor's gaze shifts to a position where the dot is not about to appear. Normally, a user's performance is quicker in the second situation compared with the first situation, and slower in the third situation compared with the first situation. This is a task which children with ASD typically fail. All these stimuli require movement responses, which provide very precise measurements of the behavioural response from the child.

The present invention can extend more broadly into the research field, particularly in the field of movement, for example the indicative characteristics that can be identified could be used to assess response to certain treatments or experimental treatments to thereby determine intended and unintended movement based effects.

The present invention has significant potential for use in the education field.

Further, the invention can be utilised for ongoing monitoring support/treatment options that may be available to a patient.

1. Apparatus for the detection and/or assessment of neurodevelopmental disorders of a user, the apparatus comprising:
control means for providing a goal-orientated task to a display, said task being formulated to elicit a movement from a user in response to said task; and
input means via which a user can input a response to said task;
wherein said control means records a user's response as multi-dimensional input data, which can be used to profile the user for the presence or absence of a neurodevelopmental disorder, the task being specifically formulated such that indicative characteristics of a user's movement over time can be identified by said multi-dimensional input data.

2. (canceled)

3. Apparatus of claim 1, wherein the control means records a user's response as two-dimensional input.

4. Apparatus of claim 1, wherein the task is formulated to elicit a three-dimensional movement by a user.

5. Apparatus of claim 1, wherein the task is formulated to elicit a two-dimensional movement by a user.

6. Apparatus of claim 1 wherein the control means comprises a computer.

7. Apparatus of claim 6 wherein the control means comprises a tablet laptop computer.

8. Apparatus of claim 1 wherein the display comprises a computer monitor or television screen.

9. Apparatus of claim 1 wherein the display comprises a LCD or plasma screen.

10. Apparatus of claim 1 wherein the input means is a hand-held manipulandum.

11. Apparatus of claim 10 wherein the hand-held manipulandum is a mouse, a stylus or a joystick.

12. Apparatus of claim 1 wherein the input means and the display are the same, and comprise an interactive tablet-screen display.

13. Apparatus of claim 1, comprising more than one input means.

14. Apparatus of claim 1, wherein said control means comprises a database comprising normative data collected from a plurality of users' previous responses to tasks using an apparatus of claim 1, said normative data being for comparison with data generated by a user of an apparatus of claim 1 for the purpose of diagnosing a neurodevelopmental disorder.

15. Apparatus of claim 1, wherein said indicative characteristics are based on a library of known calibrated results from tasks provided to children having known disorders.

16. Apparatus of claim 1, wherein the control means generates further tasks on the basis of the user's response to an initial task or tasks.

17. Apparatus for the detection and/or assessment of neurodevelopmental disorders in children, the apparatus comprising a display for displaying objective orientated information; input means for allowing input of information by a user for reaching an objective;
and control means for assessing, from characteristics of said input information in collaboration with said objective orientated information, a type of neurodevelopmental disorder.

18. (canceled)

19. A method of assessing the presence of a neurological disorder in a child, which comprises the use of an apparatus of claim 1.

20. Apparatus of claim 1, wherein the control means records a user's response as three-dimensional input.
